(19) United States
(12) Patent Application Publication
(10) Pub. No.: US 2015/0272489 A1
(43) Pub. Date: Oct. 1, 2015

(54) DEVICE, SYSTEM AND METHOD FOR TUMOR DETECTION AND/OR MONITORING

(71) Applicant: KONINKLIJKE PHILIPS N.V., EINDHOVEN (NL)

(72) Inventors: Ihor Olehovych KIRENKO, Veldhoven (NL); Gerard DE HAAN, Helmond (NL)

(21) Appl. No.: 14/669,026

(22) Filed: Mar. 26, 2015

Related U.S. Application Data
(60) Provisional application No. 61/972,502, filed on Mar. 31, 2014.

(30) Foreign Application Priority Data
Mar. 31, 2014 (EP) 14162629.1

(51) Int. Cl. A61B 5/1455; A61B 5/00; A61B 5/103

(52) U.S. Cl. CPC A61B 5/14551; A61B 5/1032; A61B 5/4887; A61B 5/7275

(57) ABSTRACT

A device, system and method for tumor detection and/or monitoring is proposed. The proposed device comprises a first analysis unit for analyzing the spatial distribution of the photoplethysmographic, PPG, amplitude of PPG signals obtained from a region of interest, a second analysis unit for analyzing the spatial distribution of arterial blood oxygen saturation obtained from said PPG signals, and an evaluation unit for detecting and/or monitoring a tumor in said region of interest based on said two analyses.

### CROSS REFERENCE TO RELATED APPLICATIONS

This application claims the benefit of U.S. provisional application Ser. No. 61/972,502 filed Mar. 31, 2014 and EP provisional application serial no. 14162629.1 filed Mar. 31, 2014, both of which are incorporated herein by reference.

### FIELD OF THE INVENTION

The present invention relates to a device, system and method for tumor detection and/or monitoring, in particular for in vivo detection and/or monitoring of a cancer tumor of a subject, such as a person or animal.

### BACKGROUND OF THE INVENTION

In the paper of Yang J, Staples O, Thomas L W, Briston T, Robson M, Poon E, Simöes M L, El-Emir E, Buffa F M, Ahmed A, Annear N P, Shukla D, Pedley B R, Maxwell P H, Harris A L, Ashcroft M., “Human CHCHD4 mitochondrial proteins regulate cellular oxygen consumption rate and metabolism and provide a critical role in hypoxia signaling and tumor progression”, J Clin Invest. 2012 February; 122(2):600-11; doi: 10.1172/JCI58780; Epub 2012 Jan. 3 an important part of oxygen-sensing machinery of tumor cells is described, which may be an early step towards a new way to treat cancer. As tumors rapidly grow and expand, the network of blood vessels bringing oxygen to their cells cannot keep up, leaving some cells starved of oxygen, or ‘hypoxic’. This would kill normal cells, but cancer cells have evolved to beat these conditions by switching on a protein called hypoxia-inducible factor (HIF), which in turn switches on other molecules inside the cell.

This cascade, called the HIF response, encourages new blood vessels to grow around and into the tumor. It also helps the tumor to adapt to hypoxic conditions by using alternative methods to produce energy.

In one of the latest studies, supported by the National Cancer Institute, smaller tumors based on magnetic resonance imaging were found to be significantly better oxygenated than larger ones. This confirmed previous investigations that show a range of hypoxic environments depending on the size of the tumor.

Tumor cells that are able to thrive despite low oxygen levels are more likely to be resistant to treatment. Previous research has shown that targeting the HIF response can block tumor growth and spread, and improves the effect of drugs that halt the growth of new blood vessels (so-called ‘anti-angiogenics’), so these results could hold promise for more effective cancer treatments in the future.

US 2004/039268 A1 discloses a system and method for quantifying the dynamic response of a target system. A time series of optical tomography data is obtained for a target tissue site in a human (or animal), using an optical wavelength, such as near infrared, at which hemoglobin is absorptive, to observe properties of the vasculature of the human. The data may be compared to baseline data of a corresponding tissue site, e.g., from a healthy human, or from another, corresponding tissue site of the human. For example, a suspected cancerous breast of a human may be compared to a known healthy breast to detect differences in the vasculature. Measures may be made of flow, oxygen supply/demand imbalance, and evidence of altered regulation of the peripheral effector mechanism. The function of the target tissue site may be analyzed, along with the coordinated interaction between multiple sites of the target system.

US 2013/274610 A1 discloses a method for visualization of cardiovascular pulsation waves. A living body is illuminated with light penetrating through a skin of the body for interacting via absorption and/or scattering with a vascular system of the living body. Light reflected from the living body is collected in a focused frame into an image capturing device. A series of frames is captured by the image capturing unit. The frames of the series of frames are multiplied by a reference function synchronized with a periodical physiological process of the body. A correlation image is formed by summarizing respective pixels over the frames of the series of frames to the reference function. An output image representing dynamics of blood-pulsation waves in the living body is calculated from the correlation images as function of the phase of the periodical physiological process of the body.

### SUMMARY OF THE INVENTION

It an object of the present invention to provide a device, system and method for automatic, unobtrusive, quick, reliable and objective tumor detection and/or monitoring.

In a first aspect of the present invention a device for tumor detection and/or monitoring is presented, the device comprising:
an interface configured to receive input signals representing electromagnetic radiation reflected from a subject at at least two different wavelengths in the range between 200 nm and 1200 nm,
a signal extraction unit configured to extract photoplethysmographic, PPG, signals from a region of interest from said input signals,
a first analysis unit configured to analyze the spatial distribution of the PPG amplitude of PPG signals obtained from said region of interest,
a second analysis unit configured to analyze the spatial distribution of arterial blood oxygen saturation obtained from said PPG signals, and
an evaluation unit configured to detect and/or monitor a tumor in said region of interest based on said two analyses.

In a further aspect of the present invention a corresponding method is presented.

In still a further aspect of the present invention a system for tumor detection and/or monitoring is presented, the system comprising:
a detection unit configured to detect electromagnetic radiation reflected from a subject at at least two different wavelengths in the range between 200 and 1200 nm, and
a device as disclosed herein for tumor detection and/or monitoring.

In yet further aspects of the present invention, there are provided a computer program which comprises program code means for causing a computer to perform the steps of the method disclosed herein when said computer program is carried out on a computer as well as a non-transitory computer-readable recording medium that stores therein a computer program product, which, when executed by a processor, causes the method disclosed herein to be performed.

Preferred embodiments of the invention are defined in the dependent claims. It shall be understood that the claimed methods, processor, computer program and medium have similar and/or identical preferred embodiments as the claimed system and as defined in the dependent claims.

The present invention is based on the idea to detect the precise location of cancer tumors in tissue and/or to monitor the development of cancer tumors based on analysis of local changes in blood vessels or microcirculation and changes of local arterial blood oxygen saturation (SpO2). The spatial analysis of blood pulsatility (generally also referred to as PPG imaging) in combination with spatial imaging of SpO2 changes is used to enable an improved diagnosis of cancer and the monitoring of a treatment effect. In other words, changes in amplitude of the pulsatile arterial blood around a tumor and the difference in dynamics of changes in SpO2 (arterial blood oxygenation) around healthy and cancer tissues are measured according to the present invention. Neither DC levels of blood volume, nor the tissue hemoglobin state, nor any 3D images are thus obtained according to the general idea of the present invention, but rather the spatial distribution of pulsatile arterial blood and its oxygenation is used for the desired tumor detection and/or monitoring.

The present invention evaluates plethysmographic (PPG) signals. Photoplethysmography (PPG) is an optical measurement technique that evaluates a time-variant change of light reflectance or transmission of an area or volume of interest. PPG is based on the principle that blood absorbs light more than surrounding tissue, so variations in blood volume with every heart beat affect transmission or reflectance correspondingly. Besides information about the heart rate, a PPG waveform can comprise information attributable to further physiological phenomena such as the respiration. By evaluating the transmittance and/or reflectivity at different wavelengths (typically red and infrared), the blood oxygen saturation can be determined.

Conventional pulse oximeters (also called contact PPG device herein) for measuring the heart rate and the (arterial) blood oxygen saturation (also called SpO2) of a subject are attached to the skin of the subject, for instance to a fingertip, earlobe or forehead. Therefore, they are referred to as ‘contact’ PPG devices. A typical pulse oximeter comprises a red LED and an infrared LED as light sources and one photodiode for detecting light that has been transmitted through patient tissue. Commercially available pulse oximeters quickly switch between measurements at a red and an infrared wavelength and thereby measure the transmittance of the same area or volume of tissue at two different wavelengths. This is referred to as time-division-multiplexing. The transmittance over time at each wavelength gives the PPG waveforms for red and infrared wavelengths. Although contact PPG is regarded as a basically non-invasive technique, contact PPG measurement is often experienced as being unpleasant and obtrusive, since the pulse oximeter is directly attached to the subject and any cables limit the freedom to move and might hinder a workflow.

Recently, non-contact, remote PPG (rPPG) devices (also called camera rPPG device herein) for unobtrusive measurements have been introduced. Remote PPG utilizes light sources or, in general radiation sources, disposed remotely from the subject of interest. Similarly, also a detector, e.g., a camera or a photo detector, can be disposed remotely from the subject of interest. Therefore, remote photoplethysmographic systems and devices are considered unobtrusive and well suited for medical as well as non-medical everyday applications.

Verkruysse et al., “Remote plethysmographic imaging using ambient light”, Optics Express, 16(26), 22 Dec. 2008, pp. 21434-21445 demonstrates that photoplethysmographic signals can be measured remotely using ambient light and a conventional consumer level video camera, using red, green and blue colour channels.

Wieringa, et al., “Contactless Multiple Wavelength Photoplethysmographic Imaging: A First Step Toward “SpO2 Camera” Technology,” Ann. Biomed. Eng. 33, 1034-1041 (2005), discloses a remote PPG system for contactless imaging of arterial oxygen saturation in tissue based upon the measurement of plethysmographic signals at different wavelengths. The system comprises a monochrome CMOS-camera and a light source with LEDs of three different wavelengths. The camera sequentially acquires three movies of the subject at the three different wavelengths. The pulse rate can be determined from a movie at a single wavelength, whereas at least two movies at different wavelengths are required for determining the oxygen saturation. The measurements are performed in a darkroom, using only one wavelength at a time.

Using PPG technology, vital signs can be measured, which are revealed by minute light absorption changes in the skin caused by the pulsating blood volume, i.e. by periodic color changes of the human skin induced by the blood volume pulse. The present invention uses PPG technology to obtain information on the spatial distribution of the PPG amplitude and the spatial distribution of SpO2, which information is then used to detect and/or monitor a tumor in a region of interest.

According to embodiments of the present invention the device further comprises an interface configured to receive input signals representing electromagnetic radiation reflected from a subject at at least two different wavelengths in the range between 200 and 1200 nm, and a signal extraction unit configured to extract photoplethysmographic, PPG, signals from said input signals. Thus, an image-based approach is used for obtaining the PPG signals, as is conventionally used for obtaining vital signs of a patient using remote PPG technology. Preferably, an imaging unit, such as a camera (e.g. an external video camera or an endoscope camera), is used for obtaining the electromagnetic radiation, in particular in the form of a set of image frames. The use of an external camera for contactless data acquisition is unobtrusive and inexpensive and can be continuously applied if needed. In other embodiments one or more pulse oximeters sensors may be used for acquiring reflected electromagnetic radiation representing PPG signals.

In a preferred embodiment said first analysis unit is configured to detect locations with higher PPG amplitude than other locations. Said locations with higher PPG amplitude indicate high blood pulsatility, which might be caused by new blood vessels formed around and into cancer tumors so that the detection of such locations indicates the presence of a tumor.

In another preferred embodiment said second analysis unit is configured to analyze the spatial distribution of arterial blood oxygen saturation over time. Said second analysis unit is particularly configured to detect locations showing a dynamic of changes of the arterial blood oxygen saturation different from other locations. Locations in a tissue around a cancer tumor show a dynamic of SpO2 changes different from healthy tissue so that the detection of such locations indicates the presence of a tumor. In a preferred embodiment the spatial distribution of changes of arterial oxygen concentration is monitored after inducing changes of oxygen supply (e.g. by holding a breath, or by reducing oxygen content in breathing air), for which a corresponding controller and/or user interface may be provided.

In still another preferred embodiment the device further comprises a third analysis unit configured to analyze the spatial distribution of tissue oxygen saturation (StO2) obtained from said PPG signals. Said third analysis unit is particularly configured to detect locations with lower tissue oxygen saturation than other locations. Locations with low saturation indicate locations of a cancer tumor so that the additional information obtained from the analysis of tissue oxygen saturation further improves the accuracy and reliability of the detection and monitoring of tissue.

In another embodiment the device further comprises a fourth analysis unit configured to analyze the spatial uniformity of skin color. This embodiment is particularly useful for the detection of skin cancer, such as melanoma.

In another embodiment said evaluation unit is configured to evaluate the result of said analyses over time to monitor the development of a tumor over time. Further, the effect of a cancer treatment can be monitored in this way.

In an embodiment of the system said detection unit comprises an imaging unit configured to acquire a set of image frames of a subject including image information and/or one or more pulse oximeter sensors.

In an embodiment of the system an illumination unit is provided configured to illuminate a region of interest with light, preferably at one or more desired wavelengths to improve the acquisition of image data and PPG signals from the image data.

In a further embodiment of the system a polarizer is provided within or in front of the imaging unit and/or within or in front of the illumination unit. Such a polarizer reduces the effect of specular reflection on the measurements which is particularly advantageous for endoscopic applications where specular reflections may particularly appear.

According to yet another aspect a device is presented for tumor detection and/or monitoring, said device comprising an interface configured to receive input signals representing electromagnetic radiation reflected from a subject at at least two different wavelengths in the range between 200 nm and 1200 nm; and a processor configured to:
extract photoplethysmographic, PPG, signals from a region of interest from said input signals,
analyze the spatial distribution of the PPG amplitude of PPG signals obtained from said region of interest,
analyze the spatial distribution of arterial blood oxygen saturation obtained from said PPG signals, and
detect and/or monitor a tumor in said region of interest based on said two analysis.

### BRIEF DESCRIPTION OF THE DRAWINGS

These and other aspects of the invention will be apparent from and elucidated with reference to the embodiment(s) described hereinafter. In the following drawings

FIG. 1 shows a schematic diagram of a first embodiment of a system including a device according to the present invention,

FIG. 2 shows a schematic diagram of first embodiment of a device according to the present invention,

FIG. 3 shows a schematic diagram of second embodiment of a device according to the present invention,

FIG. 4 shows a schematic diagram of third embodiment of a device according to the present invention,

FIG. 5 shows a schematic diagram of a second embodiment of a system according to the present invention, and

FIG. 6 shows a schematic diagram of a third embodiment of a system according to the present invention.

### DETAILED DESCRIPTION OF THE INVENTION

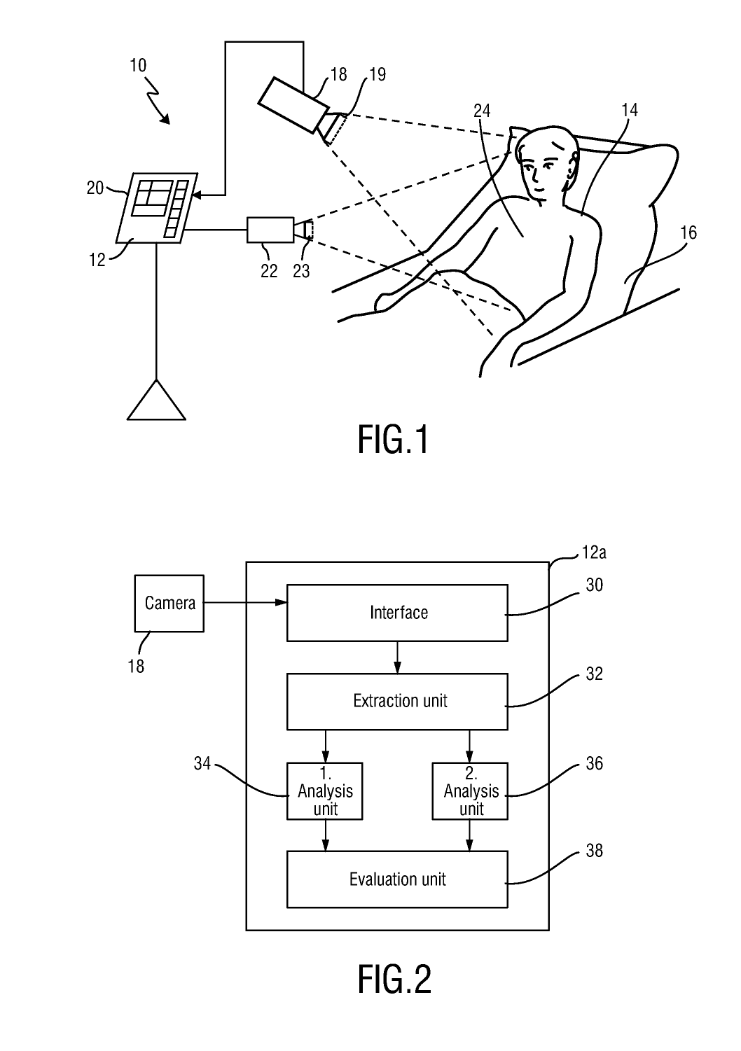

FIG. 1 shows a schematic diagram of a first embodiment of a system 10 including a device 12 for detecting and/or monitoring of a tumor of a subject 14 according to the present invention. The subject 14, in this example a patient, lies in a bed 16, e.g. in a hospital or other healthcare facility, but may also be a neonate or premature infant, e.g. lying in an incubator, or person at home or in a different environment. Image frames of the subject 14 are captured by means of a camera 18 (also generally referred to as detection unit, or as imaging unit or camera-based or remote PPG sensor) including a suitable photosensor. The camera 18 forwards the recorded image frames to the device 12, where the image frames will be processed as explained in more detail below. The device 12 preferably comprises an interface 20 for displaying the determined information and/or for providing medical personnel with an interface to change settings of the device 12 and/or other elements of the system 10. Such an interface 20 may comprise different displays, buttons, touchscreens, keyboards or other human machine interface means.

The system 10 may further comprise a light source 22 (also called illumination source), such as a lamp, for illuminating a region of interest 24, such as the skin of the patient's face or any other naked part of the body or internal tissue (e.g. using an endoscope camera unit as will be explained below), with light, for instance in a predetermined wavelength range or ranges (e.g. in the red, green and/or infrared wavelength range(s)). The light reflected from said region of interest 24 in response to said illumination is detected by the camera 18. In another embodiment no dedicated light source is provided, but ambient light is used for illumination of the subject 14. From the reflected light only light in a desired wavelength range (e.g. green light) may be detected and/or evaluated.

The system 10 may optionally further comprise one or more polarizers 19, 23 within or in front of the light source 22, the camera 18 or both to reduce the effect of specular reflection on the measurements. This is particularly advantageous for endoscopic applications where specular reflections due to high liquid levels are often pronounced.

The image frames captured by the camera 18 may particularly correspond to a video sequence captured by means of an analog or digital photosensor, e.g. in a (digital) camera. Such a camera 18 usually includes a photosensor, such as a CMOS or CCD sensor, which may also operate in a specific spectral range (visible, IR) or provide information for different spectral ranges. The camera 18 may provide an analog or digital signal. The image frames include a plurality of image pixels having associated pixel values. Particularly, the image frames include pixels representing light intensity values captured with different photosensitive elements of a photosensor. These photosensitive elements may be sensitive in a specific spectral range (i.e. representing a specific color). The image frames include at least some image pixels being representative of a skin portion of the subject. Thereby, an image pixel may correspond to one photosensitive element of a photo-detector and its (analog or digital) output or may be determined based on a combination (e.g. through binning) of a plurality of the photosensitive elements.

The uni- or bidirectional communication between the device 12, the camera 18 and the light source 22 may work via a wireless or wired communication interface, whereby it is to be noted that the light source 22 may also be configured to operate stand-alone and without communication with the device 12. Further, the device 12 and/or the light source 22 may also be incorporated into the camera 18.

A system 10 as illustrated in FIG. 1 may, e.g., be located in a hospital, healthcare facility, elderly care facility, incubator or the like. The elements of such a system are generally known in the art of vital signs monitoring using the above mentioned remote PPG technology.

FIG. 2 shows a more detailed schematic illustration of a first embodiment of a device 12a according to the present invention. The device 12a comprises an interface 30 for receiving a set of image frames of a subject (or, more generally, of input signals representing electromagnetic radiation reflected from a subject) including image information at at least two different wavelengths in the range of light, in particular in the range between 200 and 1200 nm. The interface 30 particularly receives a set of image frames acquired by the camera 18, which is generally configured for contactless detection of radiation reflected from a subject 14 in response to ambient illumination and/or illumination by the light source 22. A signal extraction unit 32 is provided for extracting photoplethysmographic (PPG) signals from a region of interest from said set of image frames. Said PPG signals are then analyzed and evaluated.

A first analysis unit 34 is provided for analyzing the spatial distribution of the PPG amplitude of PPG signals obtained from the region of interest. A second analysis unit 36 is provided for analyzing the spatial distribution of arterial blood oxygen saturation obtained from said PPG signals. The result of said two analyses is then used by an evaluation unit 38 for detecting and/or monitoring a tumor in said region of interest. The result of said evaluation may e.g. be an indication, optionally with a probability, that the examined region of interest does or does not contain a tumor.

The various units of the device 12a may be comprised in one or multiple digital or analog processors depending on how and where the invention is applied. The different units may completely or partly be implemented in software and carried out on a personal computer connected to a device for obtaining image frames of a subject, such as a camera device. Some or all of the required functionality may also be implemented in hardware, e.g. in an application specific integrated circuit (ASIC) or in a field programmable gate array (FPGA).

The first analysis unit 34 is preferably configured to measure the spatial distribution of PPG amplitude (PPG imaging) in order to detect locations with high PPG amplitude, caused by new blood vessels around and into cancer tumors.

The second analysis unit 36 is preferably configured to detect locations showing a dynamic of changes of the arterial blood oxygen saturation (SpO2) different from other locations. Locations in a tissue around cancer tumor would have a dynamic of SpO2 changes different from healthy tissue.

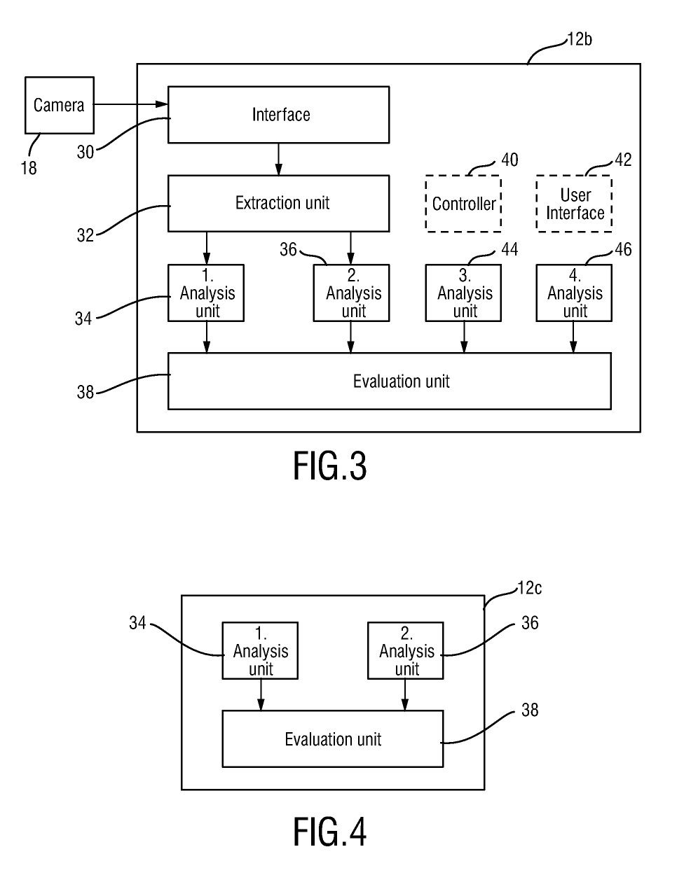

In a preferred embodiment, an SpO2 map is obtained by the second analysis unit. This SpO2 map, in particular changes of this SpO2 map, are analyzed by inducing changes of oxygen supply (e.g. by holding a breath, or by reducing oxygen content in breathing air) and monitoring the spatial distribution of changes of SpO2. For this purpose, as exemplarily shown in the second embodiment of the device 12b depicted in FIG. 3, a controller 40 and/or user interface 42 for inducing changes of oxygen supply to the subject 14 may be provided. For instance, the controller 40 may control the oxygen content in the breathing air provided to the subject 14 (e.g. via a facial mask). Additionally or alternatively, the user interface may provide instructions to the subject 14 for controlled breathing, e.g. to hold the breath for some time and to deeply inhale after said time.

The present invention uses an analysis of spatial non-uniformity of SpO2 changes. Therefore, in order to provide such analysis, SpO2 needs to be changed. Such changes might be just normal (healthy) variations of arterial oxygenation (e.g. due to physical exercise) or may be induced artificially by reducing an oxygen supply temporally (holding the breath, reducing the oxygen saturation of the air). Thus, any method which temporally reduces the supply of oxygen can be used for obtaining the SpO2 map.

The second embodiment of the device 12b further comprises a third analysis unit 44 for analyzing the spatial distribution of tissue oxygen saturation obtained from said PPG signals. In particular, locations with lower tissue oxygen saturation than other locations are detected. Preferably, an StO2 map is obtained by measuring the spatial distribution of StO2 and detecting locations with low saturation, which will correspond to cancer tumor locations.

Additionally or alternatively, a fourth analysis unit 46 is provided for analyzing the spatial uniformity of skin color from said PPG signals, in particular to measure the color DC levels, which is particularly useful in the detection of skin cancer, such as melanoma. Symptoms of skin cancer, such as melanoma, are a change in size, shape, color of a mole and/or other skin growth, such as a birthmark. Melanoma may appear as a new mole. However, often this approach for diagnosis lacks specificity. This embodiment of the present invention thus provides an improvement of specificity by combining SpO2 and PPG imaging, as discussed above, with other methods of skin cancer detection based on local skin color changes.

Preferably, the evaluation unit 38 is configured to evaluate the result of said analyses over time to monitor the development of a tumor over time. This allows not only to detect a tumor but also to monitor the development (e.g. the change of the size and/or form) of the tumor and the progress of cancer treatment.

The acquisition of the input information to the first and second analysis units 34, 36, i.e. the acquisition of PPG signals, may also be made differently from the above described embodiment. The PPG signals may also be acquired in advance and stored in a memory for later analysis and evaluation. Hence, a third, more general embodiment of a device 12c, as schematically depicted in FIG. 4, may only comprise the first analysis unit 34, the second analysis unit 36 and the evaluation unit 38 as described above. The PPG signals are then directly provided to the analysis units 34, 36 for processing.

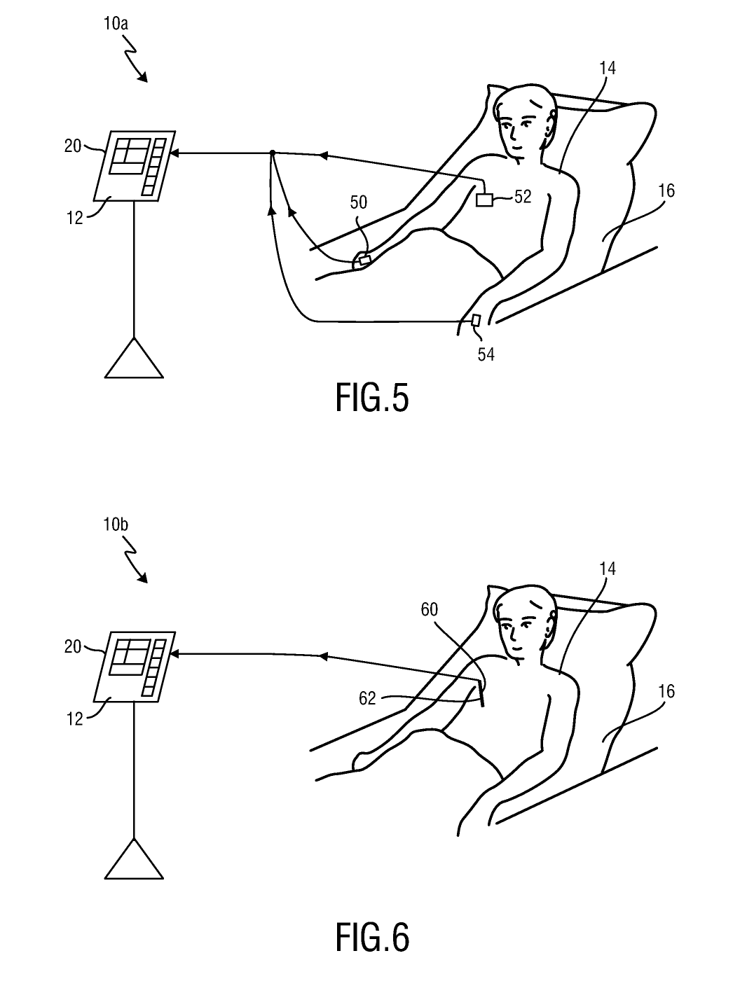

Another embodiment of a system 10a is schematically depicted in FIG. 5. Instead of an imaging unit and an illumination unit it comprises one or more contact pulse oximeter sensors 50, 52, 54 placed at the subject's body for obtaining the PPG signals representing electromagnetic radiation reflected from skin of the subject 12, in particular in the red and infrared wavelength ranges. Said pulse oximeter sensors 50, 52, 54 are preferably similar or identical to conventional sensors used for obtaining SpO2 information in reflective mode. The device 12 may be configured as the third embodiment shown in FIG. 4 since the sensors 50, 52, 54 may directly provide the PPG signals. Thus, generally the same principle as discussed above for use with PPG signals derived from image data acquired by a camera (i.e. in a contactless way) can be used with PPG signals obtained by pulse oximeter sensors (i.e. in a contact way).

Generally, the present invention can be applied for detection and/or monitoring of tumor at any internal or external body tissue. FIG. 6 shows still another embodiment of a system 10b. In this embodiment an endoscope 60 carrying a camera 62 as imaging unit and, optionally, an illumination source (not shown) is used for obtaining image data from within the body at an area of interest. The device 12 may be configured as shown in FIG. 2 or 3, i.e. the image data obtained by the endoscope camera 62 are evaluated in substantially the same manner as explained above.

The proposed system, device and method are thus configured to detect abnormalities in the spatial distribution of at least two parameters from the above mentioned parameters, which are specific for cancer tumor. The effect of a cancer treatment can be monitored by objectively estimating changes in parameters of PPG imaging, SpO2 map and/or StO2 map around spatial location of a cancer tumor, particularly in comparison with healthy tissue. The information of PPG imaging, SPO2 map and optionally StO2 map and DC distribution is preferably gathered by a camera, with at least two wavelengths in a visible and invisible color spectrum. By way of example, the present invention can be applied in the field of health care, e.g. unobtrusive remote patient monitoring and general surveillance. In general, the present invention allows both spot-check and continuous monitoring. Further, the present invention can be used in perioperative care for tumor detection.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Furthermore, the different embodiments can take the form of a computer program product accessible from a computer usable or computer readable medium providing program code for use by or in connection with a computer or any device or system that executes instructions. For the purposes of this disclosure, a computer usable or computer readable medium can generally be any tangible device or apparatus that can contain, store, communicate, propagate, or transport the program for use by or in connection with the instruction execution device.

In so far as embodiments of the disclosure have been described as being implemented, at least in part, by software-controlled data processing devices, it will be appreciated that the non-transitory machine-readable medium carrying such software, such as an optical disk, a magnetic disk, semiconductor memory or the like, is also considered to represent an embodiment of the present disclosure.

The computer usable or computer readable medium can be, for example, without limitation, an electronic, magnetic, optical, electromagnetic, infrared, or semiconductor system, or a propagation medium. Non-limiting examples of a computer readable medium include a semiconductor or solid state memory, magnetic tape, a removable computer diskette, a random access memory (RAM), a read-only memory (ROM), a rigid magnetic disk, and an optical disk. Optical disks may include compact disk-read only memory (CD-ROM), compact disk-read/write (CD-R/W), and DVD.

Further, a computer usable or computer readable medium may contain or store a computer readable or usable program code such that when the computer readable or usable program code is executed on a computer, the execution of this computer readable or usable program code causes the computer to transmit another computer readable or usable program code over a communications link. This communications link may use a medium that is, for example, without limitation, physical or wireless.

A data processing system or device suitable for storing and/or executing computer readable or computer usable program code will include one or more processors coupled directly or indirectly to memory elements through a communications fabric, such as a system bus. The memory elements may include local memory employed during actual execution of the program code, bulk storage, and cache memories, which provide temporary storage of at least some computer readable or computer usable program code to reduce the number of times code may be retrieved from bulk storage during execution of the code.

Input/output, or I/O devices, can be coupled to the system either directly or through intervening I/O controllers. These devices may include, for example, without limitation, keyboards, touch screen displays, and pointing devices. Different communications adapters may also be coupled to the system to enable the data processing system to become coupled to other data processing systems, remote printers, or storage devices through intervening private or public networks. Non-limiting examples are modems and network adapters and are just a few of the currently available types of communications adapters.

The description of the different illustrative embodiments has been presented for purposes of illustration and description and is not intended to be exhaustive or limited to the embodiments in the form disclosed. Many modifications and variations will be apparent to those of ordinary skill in the art. Further, different illustrative embodiments may provide different advantages as compared to other illustrative embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated. Other variations to the disclosed embodiments can be understood and effected by those skilled in the art in practicing the claimed invention, from a study of the drawings, the disclosure, and the appended claims.

1. A device for tumor detection and/or monitoring, said device comprising:
an interface configured to receive input signals representing electromagnetic radiation reflected from a subject at at least two different wavelengths in the range between 200 nm and 1200 nm,
a signal extraction unit configured to extract photoplethysmographic, PPG, signals from a region of interest from said input signals
a first analysis unit configured to analyze the spatial distribution of the PPG amplitude of PPG signals obtained from said region of interest,
a second analysis unit configured to analyze the spatial distribution of arterial blood oxygen saturation obtained from said PPG signals, and
an evaluation unit configured to detect and/or monitor a tumor in said region of interest based on said two analyses.

2. The device as claimed in claim 1,
wherein said first analysis unit is configured to detect locations with higher PPG amplitude than other locations.

3. The device as claimed in claim 1,
wherein said second analysis unit is configured to analyze the spatial distribution of arterial blood oxygen saturation over time.

4. The device as claimed in claim 3,
wherein said second analysis unit is configured to detect locations showing a dynamic of changes of the arterial blood oxygen saturation different from other locations.

5. The device as claimed in claim 3,
further comprising a controller and/or user interface configured to induce changes of oxygen supply to the subject.

6. The device as claimed in claim 1,
further comprising a third analysis unit configured to analyze the spatial distribution of tissue oxygen saturation obtained from said PPG signals.

7. The device as claimed in claim 6,
wherein said third analysis unit is configured to detect locations with lower tissue oxygen saturation than other locations.

8. The device as claimed in claim 1,
further comprising a fourth analysis unit configured to analyze the spatial uniformity of skin color.

9. The device as claimed in claim 1,
wherein said evaluation unit is configured to evaluate the result of said analyses over time to monitor the development of a tumor over time.

10. A method for tumor detection and/or monitoring, said method comprising:
receiving input signals representing electromagnetic radiation reflected from a subject at at least two different wavelengths in the range between 200 nm and 1200 nm,
extracting photoplethysmographic, PPG, signals from a region of interest from said input signals,
analyzing the spatial distribution of the PPG amplitude of PPG signals obtained from said region of interest,
analyzing the spatial distribution of arterial blood oxygen saturation obtained from said PPG signals, and
detecting and/or monitoring a tumor in said region of interest based on said two analyses.

11. A system for tumor detection and/or monitoring, said system comprising:
a detection unit configured to detect electromagnetic radiation reflected from a subject at at least two different wavelengths in the range between 200 and 1200 nm, and
a device as claimed in claim 1 for tumor detection and/or monitoring.

12. The system as claimed in claim 11,
wherein said detection unit comprises an imaging unit configured to acquire a set of image frames of a subject including image information and/or one or more pulse oximeter sensors.

13. The system as claimed in claim 11,
further comprising a polarizer within or in front of the imaging unit.

14. A computer readable non-transitory medium having instructions stored thereon which, when carried out on a computer, cause the computer to perform the steps of the method as claimed in claim 10.

15. A device for tumor detection and/or monitoring, said device comprising an interface configured to receive input signals representing electromagnetic radiation reflected from a subject at at least two different wavelengths in the range between 200 nm and 1200 nm; and a processor configured to:
extract photoplethysmographic, PPG, signals from a region of interest from said input signals,
analyze the spatial distribution of the PPG amplitude of PPG signals obtained from said region of interest,
analyze the spatial distribution of arterial blood oxygen saturation obtained from said PPG signals, and
detect and/or monitor a tumor in said region of interest based on said two analysis.
